(19) United States
(12) Patent Application Publication
(10) Pub. No.: US 2012/0101515 A1
(43) Pub. Date: Apr. 26, 2012

(54) PRESSURE INDUCED COLOR CHANGE FOR BALLOON CATHETER CHASSIS

(75) Inventor: Leonard Barbod, San Diego, CA (US)

(73) Assignee: ABBOTT CARDIOVASCULAR SYSTEMS INC., Santa Clara, CA (US)

(21) Appl. No.: 12/909,635

(22) Filed: Oct. 21, 2010

(51) Int. Cl. A61M 25/10

(52) U.S. Cl. 606/194

(57) ABSTRACT

A balloon catheter has a visual indicator to indicate when a maximum pressure has been reached or exceeded for forensic purposes after a catheter has failed. The visual indicator can be mechanical, such as a physical member that stretches or discolors upon yielding. Or the visual indicator can be the use of a force sensitive polymer that changes color upon a predetermined stress, such as for example mechanophores. Where such visual indicators are present, a failed catheter can be investigated to determine if the failure was likely the result of overpressurization, or if the catheter failed for defects unrelated to the pressure.

### BACKGROUND OF THE INVENTION

The invention relates to the field of intravascular medical devices, and more particularly to devices for detecting and preventing over-pressurization of a balloon catheter.

In percutaneous transluminal coronary angioplasty (PTCA) procedures, a guiding catheter is advanced until the distal tip of the guiding catheter is seated in the ostium of a desired coronary artery. A guidewire, positioned within an inner lumen of a dilatation catheter, is first advanced out of the distal end of the guiding catheter into the patient's coronary artery until the distal end of the guidewire crosses a lesion or obstruction to be dilated. The dilatation catheter having an inflatable balloon on the distal portion thereof is then advanced into the patient's coronary anatomy, over the previously introduced guidewire, until the balloon of the dilatation catheter is properly positioned across the lesion or obstruction. Once properly positioned, the dilatation balloon is inflated with liquid one or more times to a predetermined size at relatively high pressures (e.g., greater than 8 atmospheres) so that the stenosis is compressed against the arterial wall and the wall expanded to open up the passageway. Generally, the inflated diameter of the balloon is approximately the same diameter as the native diameter of the body lumen being dilated so as to effect the dilatation without over-expanding the arterial wall. Substantial, uncontrolled expansion of the balloon against the vessel wall can cause trauma to the vessel wall. After the balloon is finally deflated, blood flow may resume through the dilated artery and the dilatation catheter can be withdrawn from the patient.

In such angioplasty procedures, there may be restenosis of the artery, i.e. reformation of the arterial blockage, or obstructions that cannot be resolved by inflation of the balloon alone. These conditions often necessitate either another angioplasty procedure, or some other method of repairing, strengthening, or unblocking the dilated area. To reduce the restenosis rate and to strengthen or unblock the dilated area, physicians frequently implant an intravascular prosthesis, generally called a stent, inside the artery at the site of the lesion. Stents may also be used to repair vessels having an intimal flap or dissection or to generally strengthen a weakened section of a vessel. Stents are usually delivered to a desired location within a coronary artery in a contracted condition on a balloon of a catheter which is similar in many respects to a balloon angioplasty catheter and expanded to a larger diameter by expansion of the balloon. The balloon is deflated to remove the catheter and the stent left in place within the artery at the site of the dilated lesion.

In the design of catheter balloons, balloon characteristics such as strength, flexibility and compliance must be tailored to provide optimal performance for a particular application. Angioplasty and stent delivery balloons preferably have high strength for inflation at relatively high pressure, and high flexibility and softness for improved ability to track the tortuous anatomy and cross lesions. The balloon compliance is chosen so that the balloon will have the required amount of expansion during inflation. Compliant balloons, for example balloons made from materials such as polyethylene, exhibit substantial stretching upon the application of a tensile force. Noncompliant balloons, for example balloons made from materials such as PET, exhibit relatively little stretching during inflation, and therefore provide controlled radial growth in response to an increase in inflation pressure within the working pressure range. However, noncompliant balloons generally have relatively low flexibility and softness, making it challenging to provide a low compliant balloon with high flexibility and softness for enhanced catheter trackability. A compromise is typically struck between the competing considerations of softness/flexibility and noncompliance, which, as a result, has limited the degree to which the compliance of catheter balloons can be further lowered.

The latest generation of balloon and stent delivery systems provides the most deliverable, lowest profile devices yet for cardiologists to treat patients. Cardiologists are able to access and deliver balloons to the most distal, challenging legion types than ever before. This has led to an increasing demand for higher and higher balloon and stent delivery systems balloon rated burst pressures. Often the desired pressures exceed twenty (20) atmospheres.

In this procedure, cardiologists very often inflate the balloon and stent catheters beyond their rated burst pressure. This is usually done to achieve a better PTCA result for the patient and the physicians rely on the rated burst pressure to have a known factor of safety incorporated therein. It is widely held that the risk is low for small increases over the rated burst pressure. Because of this prevalence to overinflate the balloons, when a balloon does burst it is difficult for the manufacturer to evaluate whether there was a flaw in the design or whether the balloon was simply over-inflated by the physician.

Further, balloon and stent delivery systems are designed from a safety stand point to fail, under hydraulic pressure, at the balloon and not the chassis. There is a considerably higher patient risk associated with catheter chassis failures for rupture compared to balloon burst failures. This requires the chassis to be designed to statistically withstand a significantly higher burst pressure compared with balloons. This design requirement can limit the material choices and dimensions needed for best catheter deliverability and low profile performance. The latest multiple layer balloon technologies allow for even higher rated burst pressures, which challenges the current technology to create chasses able to withstand even higher burst pressures and maintain safety needed for the balloon to fail before the chassis.

### SUMMARY OF THE INVENTION

The present invention is directed to devices and methods for detecting over-pressurization of a catheter, either in situ or post-procedure. In situ devices can alert a physician to the possible risk of rupture of the catheter, which post-procedure devices allow a forensics evaluation of why a catheter may have failed during an operation. For example, in one preferred embodiment, a catheter incorporates a force-sensitive polymer that can change color when mechanically stressed to form a catheter chassis. In some stress-induced color changing materials, force-sensitive polymers contain mechanically active molecules called mechanophores. When stretched with a certain force, specific chemical reactions are triggered in the mechanophores. Mechanophores contain molecules called spiropyrans capable of vivid color changes when they undergo mechanically induced stress. Normally colorless, the spiropyran turns, for example, red or purple when exposed to certain levels of mechanical stress. Other types of mechanophores can also be used. This material can be used within or in conjunction with a catheter's luer assembly or catheter chassis, and the mechanophores are selected so as to indicate (i.e., color change) above a predetermined internal pressure on the catheter. If the mechanophore was selected to indicate above the rated burst pressure of the catheter and the catheter failed, a forensic review of the catheter would quickly determine whether the failure was due to the cardiologist over-pressurizing the catheter beyond the rated burst pressure or whether the catheter failed for some other reason. This would provide invaluable feedback to both the practitioner as well as the catheter's manufacturer or complaint investigation team regarding the cause of the failure as well as ways to correct the problem.

Alternatively, existing polymers may be used to form structure that will mechanically deform at a certain internal pressure of the catheter. For example, a portion of the catheter luer that is exposed to the chassis internal pressure and located within view of the cardiologist during catheter balloon inflation can be designed to physically deform under a specified pressure. The structure could have a wall thickness or wall section designed to deform above a selected pressure. The deformation could be a permanent deformation caused by exceeding the yield point of the material and this could also cause discoloration. The inflation/deflation and operation of the catheter would not ordinarily be compromised by this deformation in a preferred embodiment. There could also be a tactile deformation that could be felt as well as observed.

A third embodiment involves the use of a pressure relief valve that is designed to open and relieve applied pressure above a certain material stress. The valve would close and maintain inflation/deflation integrity below the designated maximum material stress. The valve would also preferably include a visual indication that the relief valve was actuated, alerting the physician and/or the manufacturer in the case where there was a failure of the catheter or for collecting data about the procedure.

### BRIEF DESCRIPTION OF THE DRAWINGS

FIG. 1 is an elevational view, partially in section, of an over-the-wire type stent delivery balloon catheter embodying features of the invention.

FIGS. 2 and 3 are transverse cross sectional views of the catheter of FIG. 1, taken along lines 2-2 and 3-3, respectively.

FIG. 4 is a modified balloon inflation luer with an inflation tube adaptor coupled thereto;

FIG. 5 is the modified balloon inflation luer of FIG. 4 with a visual indicator mounted thereon;

FIG. 6 is the modified balloon inflation luer of FIG. 4 with a relief valve incorporated thereon;

FIG. 7 is a visual indicator that can be mounted onto balloon inflation luer to indicate over-pressurization;

FIG. 8 is another over-pressurization indicator device that can be implemented into the balloon catheter inflation system;

FIG. 9 is yet another visual indicator of over-pressurization that can be implemented into the balloon catheter inflation system;

FIG. 10 is another visual indicator comprising a tubing that is exposed to the internal pressure of the inflation lumen and expands when overpressurized;

FIG. 11 is yet another physical indicator of over-pressurization that can be incorporated into the balloon catheter inflation system; and

FIG. 12 is still another physical indicator of over-pressurization that can be incorporated into the balloon catheter inflation system.

### DETAILED DESCRIPTION OF THE PREFERRED EMBODIMENTS

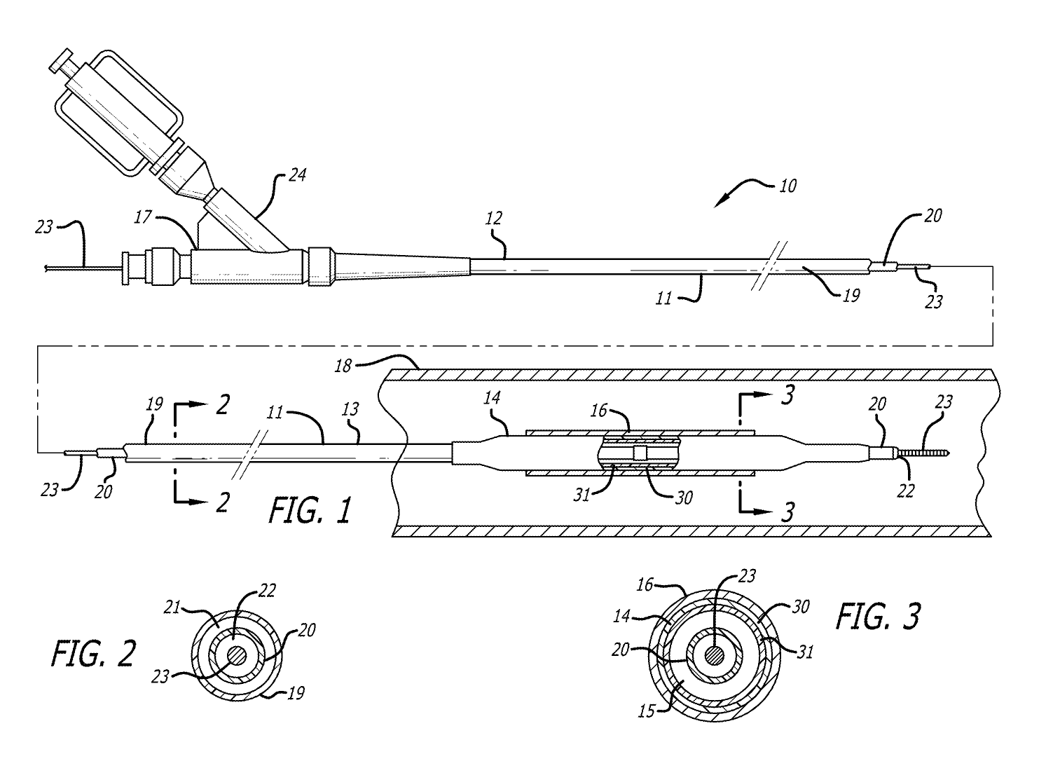

FIG. 1 illustrates a stent delivery balloon catheter 10 which embodies features of the invention, generally comprising an elongated catheter shaft 11 having a proximal shaft section 12, a distal shaft section 13, an inflation lumen 21, and a guidewire lumen 22 configured to slidably receive a guidewire 23 therein, and having a balloon 14 mounted on the distal shaft section. An inflation and guidewire dual port adaptor 17 is located on a proximal end of the catheter shaft and provides access to the guidewire lumen 22, and has an inflation luer 24 configured for connecting to a source of inflation fluid (not shown). FIG. 1 illustrates the balloon in a deflated configuration for advancement within a patient's body lumen 18, which has been depicted in an idealized, enlarged manner. A radially expandable stent 16 may be releasably mounted on the balloon 14 for delivery and deployment within the body lumen 18. The balloon catheter 10 is advanced in the body lumen 18 over the guidewire 23 with the balloon 14 in the deflated configuration, and then the balloon 14 is inflated by introducing inflation fluid into the balloon interior via the luer 24 to expand the balloon and stent mounted thereon. Once the stent is expanded and in place, the balloon is then deflated to allow for repositioning or removal of the catheter from the body lumen 18, leaving the stent 16 implanted in the body lumen 18.

In the illustrated embodiment, the shaft 11 comprises an outer tubular member 19 which cooperates with an inner tubular member 20 to define the inflation lumen 21, and where the inner tubular member 20 further defines the guidewire lumen 22 as seen in FIG. 2. The balloon 14 has a proximal skirt section sealingly secured to the distal end of the outer tubular member 19, and a distal skirt section sealingly secured to a distal end of the inner tubular member 20, so that an interior 15 of the balloon is in fluid communication with the inflation lumen 21 of the shaft. FIG. 3 illustrates a transverse cross section of the catheter of FIG. 1, taken along line 3-3, although the space between the inner surface of the deflated balloon and the outer surface of the portion of the shaft 11 therein is somewhat exaggerated for ease of illustration. A variety of alternative suitable catheter shaft configurations can be used as are conventionally known.

Although not illustrated, the balloon 14 of the invention typically has a deflated configuration with wings wrapped around the balloon to form a low profile configuration for introduction and advancement within a patient's body lumen. As a result, the balloon 14 inflates to a nominal working diameter by unfolding and filling the molded volume of the balloon.

Balloon 14 has a first layer 30, and a second layer 31 which is an inner layer relative to the first layer 30. In the illustrated embodiment, the second layer 31 is on an inner surface of the balloon 14 with the first layer 30 defining an outer surface of the balloon 14 and the second layer 31 defining an inner surface of the balloon 14. However, the balloon 14 of the invention can alternatively have more or fewer layers. Additional layer(s) increase the dimensions of the tube/balloon formed therefrom to a desired value, and/or can be used to provide an inner or outer surface of the balloon with a desired characteristic. Therefore, it should be understood that the balloon 14 of the invention discussed below has at least two layers, and optionally includes one or more additional layers, unless otherwise noted as having a specified set number of layers.

A variety of suitable materials can be used to form the balloon layers 30, 31. In one embodiment, the first and second polymeric materials are elastomers providing a relatively low flexural modulus for balloon flexibility, although nonelastomers can alternatively be used. Presently preferred materials are from the same polymeric family/class such as polyamides including nylons and polyether block amides (PEBAX). Forming the layers of compatible polymeric materials allows for heat fusion bonding the layers together. The layers can alternatively be formed of different polymer classes which are not sufficiently compatible to fusion bond together, in which case a tie layer is typically provided between the outer and inner layers 30, 31 to bond the balloon layers together. For example, a PET inner layer and a PEBAX outer layer typically have a tie layer of an adhesive polymer such as Primacor (a functionalized polyolefin) therebetween. With all balloon catheters, however, it is a primary objective that the balloon, regardless of the materials used to form the balloon, should fail before the catheter chassis fails. If the balloon fails, the catheter can still be withdrawn from the patient and the procedure repeated with a new catheter. However, if the catheter chassis fails and part of the catheter becomes lodged in the patient's vascular, a potentially life threatening condition could result.

The dimensions of catheter 10 are determined largely by the size of the balloon and guidewire to be employed, the catheter type, and the size of the artery or other body lumen through which the catheter must pass or the size of the stent being delivered. Typically, the outer tubular member 14 has an outer diameter of about 0.025 to about 0.04 inch (0.064 to 0.10 cm), usually about 0.037 inch (0.094 cm), and the wall thickness of the outer tubular member 14 can vary from about 0.002 to about 0.008 inch (0.0051 to 0.02 cm), typically about 0.003 to 0.005 inch (0.0076 to 0.013 cm). The inner tubular member 16 typically has an inner diameter of about 0.01 to about 0.018 inch (0.025 to 0.046 cm), usually about 0.016 inch (0.04 cm), and a wall thickness of about 0.004 to about 0.008 inch (0.01 to 0.02 cm). The overall length of the catheter 10 may range from about 100 to about 150 cm, and is typically about 143 cm. Preferably, balloon 24 has a length about 0.8 cm to about 6 cm, and an inflated working diameter of about 2 to about 5 mm.

The various components may be joined using conventional bonding methods such as by fusion bonding or use of adhesives. Although the shaft is illustrated as having an inner and outer tubular member, a variety of suitable shaft configurations may be used including a dual lumen extruded shaft having a side-by-side lumens extruded therein. Similarly, although the embodiment illustrated in FIG. 1 is an over-the-wire type stent delivery balloon catheter, the catheter of this invention may comprise a variety of intravascular catheters, such as a rapid exchange type balloon catheter. Rapid exchange catheters generally comprise a shaft having a relatively short guidewire lumen extending from a guidewire distal port at the catheter distal end to a guidewire proximal port spaced a relatively short distance from the distal end of the catheter and a relatively large distance from the proximal end of the catheter.

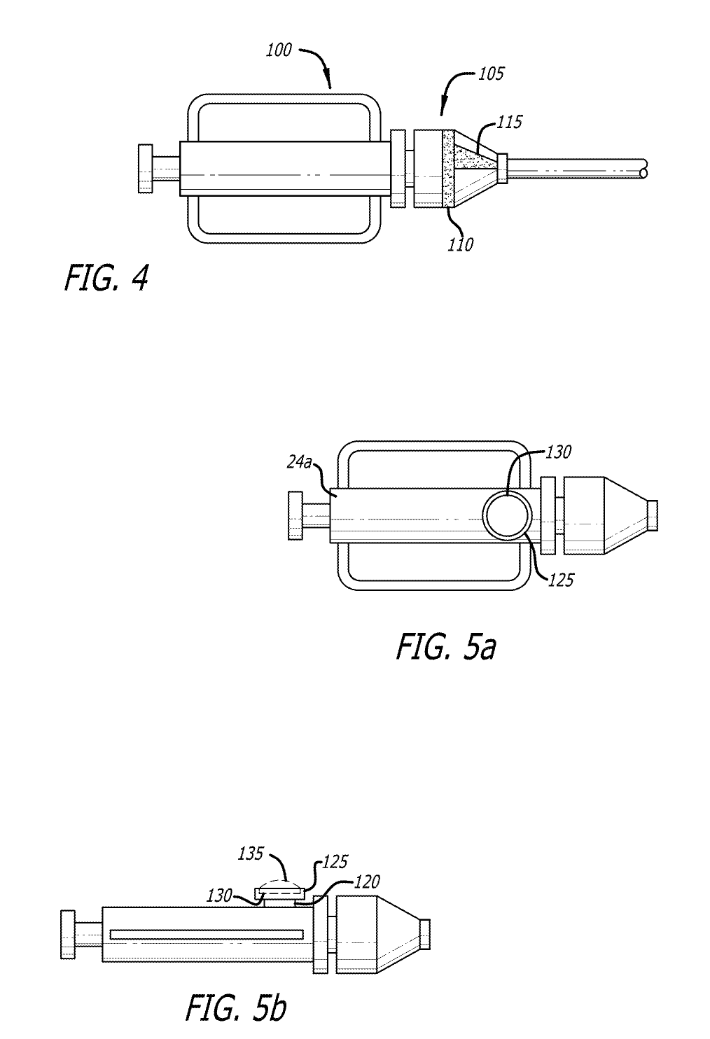

To accomplish the objects of the present invention, a first embodiment is shown in FIG. 4 in which the luer 24 is fitted with a inflation tube adaptor 100 made with a material that includes or comprises mechanophores. It should be noted that while the inflation tube adaptor 100 is a convenient location for the mechanophores, the material can also be located elsewhere on the catheter as long as it is subject to the internal pressure experienced by the balloon. The adaptor 100 is formed as a ring or tube in fluid communication with the luer 24 so as to experience the inflation pressure applied to the balloon 14. As the balloon 14 expands due to the pressure applied at the luer 24, the adapter 100 will experience a stress due to the internal pressure. This internal pressure applied stress, if it is elevated to a certain level, will cause the mechanophores to cause a color change in the material, and this color change is indicative of the level of pressure applied during the inflation process. By examining the adapter 100 after the procedure, it may be determined what level of pressure was applied during the procedure, which can then be used for forensic purposes if there were to be a failure of the balloon or catheter.

FIG. 4 also illustrates a second alternative structure that could be used in lieu of (or theoretically in addition to) the adapter 100, namely a nose piece 105 that forms a part of the luer 24 and is made of, or partially made of, mechanophores. The nose piece 105 may have a ring 110 or wedge 115 of mechanophores that can be used to evaluate internal pressure as above. Or, the entire inflation/guidewire adapter 17 and luer 24 can be made of a force sensitive material such as a mechanophores.

Alternatively, the adapter 100 or the nose piece 105 can deform mechanically as a result of the pressure without actually changing color, such as forming a bulge or bubble if the pressure becomes too high. This deformation can alert the doctor that the inflation pressure has exceeded the recommended pressure, but does not jeopardize the integrity of the catheter. In FIGS. 5a,b, the modified luer 24a includes a neck 120 in fluid communication with the luer 24a and connected to a cylindrical ring 125. The ring includes a bladder or flexible membrane 130 that reacts to the internal pressure in the inflation lumen by bulging out to form a bubble 135 when the inflation pressure is large enough. By controlling the thickness, diameter, material, etc. of the bladder 130 to expand only after reaching a certain designated pressure, the physician can have a physical indicator as the balloon 14 is being inflated that lets the physician know when the maximum pressure of the balloon catheter is approaching or exceeded. Alternatively, the bladder 130 can be made of a force sensitive material such as a mechanophore that changes color upon reaching a designated stress, alerting the doctor by a change of color that a predetermined pressure has been achieved relative to a safety or failure specification of the catheter.

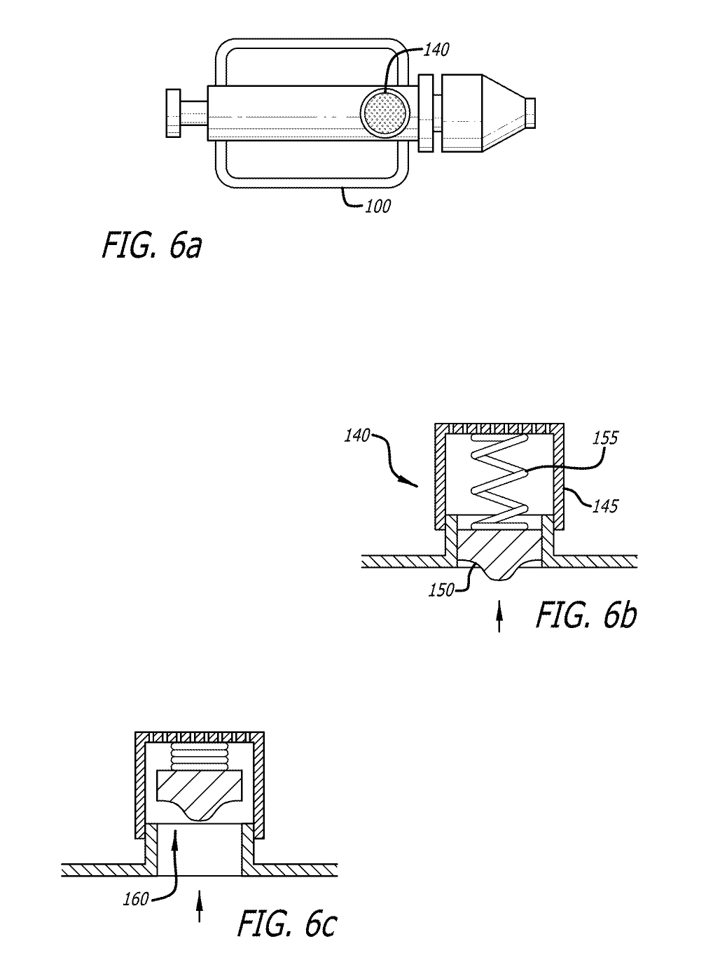

FIGS. 6a-c illustrate an alternative embodiment employing a check valve 140 as a safety measure to ensure that the maximum allowable internal pressure in the inflation lumen and balloon 14 is not exceeded. The check valve 140 is designed to allow air or gas to escape when a predetermined pressure is achieved to prevent over-pressurization. The check valve 140 includes a chamber 145 with vent holes that allow a gas to escape when it enters the chamber. A plug 150 is biased against the mouth 160 of the chamber 145 by a biasing element 155 to bias the check valve 140 in the closed position (FIG. 6b), preventing any gas used to inflate the balloon 14 from escaping through the check valve 140. When the internal pressure causes the plug 150 to recede into the chamber 145 due to the inflation pressure overcoming the force of the biasing element 155, gas escapes around the plug and through the vent holes to prevent over-pressurization. This is illustrated in FIG. 6c.

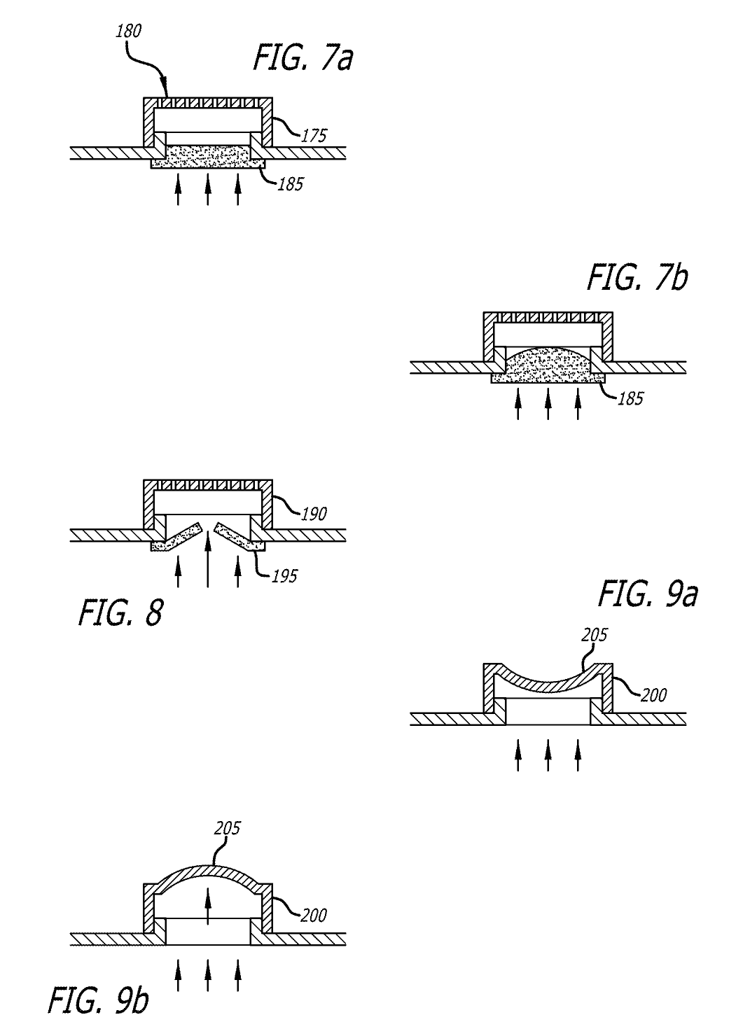

In another alternative embodiment in FIG. 7, the check valve of FIG. 6 is replaced with a button 175 having a window 180 that is connected to the luer 24, and a force sensitive pad or disk 185 is connected to the button and subject to the internal pressure of the inflation lumen. The window 180 is preferably transparent so that it may be viewed from outside the catheter while the procedure is ongoing, although the structure could also be opaque and the results of the pressure test viewed by extracting the button 175 after the procedure is over. The nominal condition is shown in FIG. 7a, where the disk is made of a force sensitive material that changes color when a specified stress is experienced. If the internal pressure of the inflation lumen does not exceed the nominal pressure, the disk 185 remains in an unchanged state. In FIG. 7b, the disk has changed color because the internal pressure of the inflation lumen has caused the disk 185 to deform and yield to the point where it has either experienced permanent deformation or in the case of a mechanophores, it has changed color indicating a predetermined stress (and therefore pressure) has been achieved. This visual indicator can be used to determine whether a catheter has failed due to overpressurization as opposed to another mechanical or manufacturing failure.

FIG. 8 discloses yet another visual indicator of over-pressurization that may be associated with a balloon catheter. In FIG. 8, a cap 190 may be attached to the catheter at, for example, the inflation luer 24. The cap 190 incorporates a mechanical seal 195 at its opening that is frangible upon exposure to a high enough internal pressure. This may be accomplished by scoring the seal or creating some break point that will separate only upon exposure to a specified internal pressure. When the internal pressure “pops” the seal 195 as shown in FIG. 8, it is irreversible in that it cannot be rejoined, and thus one can determine that a specified pressure occurred during the inflation operation.

FIGS. 9a,b show a visual pressure indicator that is reversible and therefore not especially suitable for post-procedure forensics, but rather for in-procedure monitoring of over-pressurization. A cylindrical drum 200 is attached to the catheter at the luer 24 or elsewhere where the inflation pressure is present. The drum 200 includes a flexible membrane 205 stretched over the top surface, where the membrane 205 may have a concave orientation under nominal pressure conditions. However, as shown in FIG. 9b, the membrane may bow or expand to a convex configuration where the internal pressure reaches a specified level corresponding to an over-pressurization condition. Using this embodiment, if the physician observes that the membrane 205 has transformed from a concave orientation to a convex orientation, the inflation pressure has exceeded a predetermined value preferably corresponding to a pressure just below the over-pressurization condition. Further pressurization could jeopardize the integrity of the catheter balloon and cause it to fragment or tear in the patient.

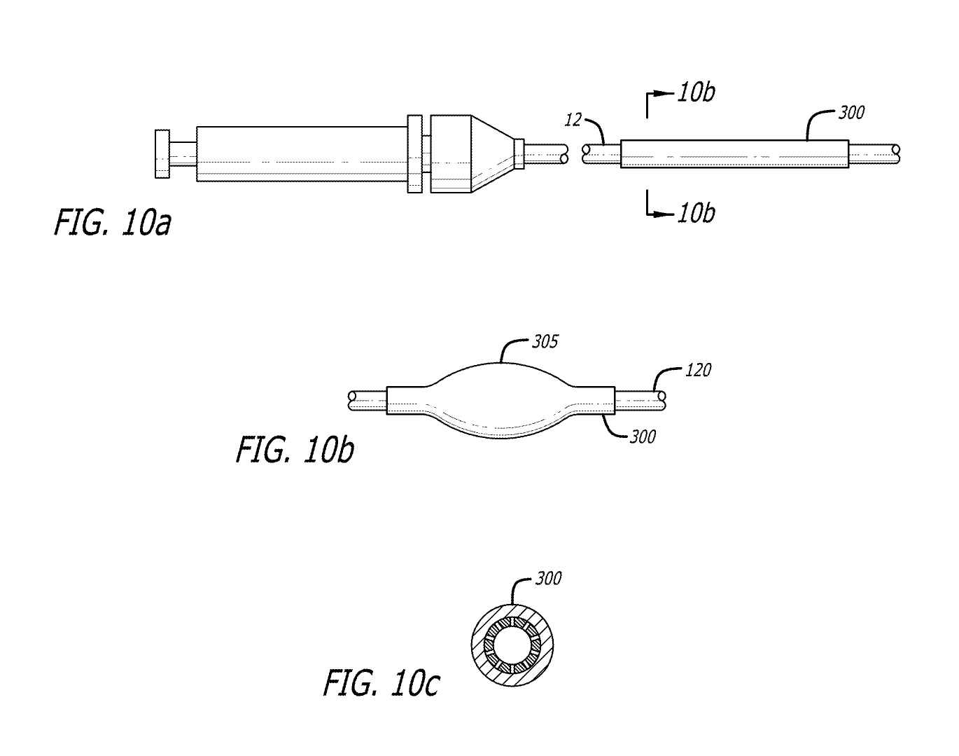

FIG. 10a illustrates another embodiment in which a pressure sensitive tube 300 is located over the proximal shaft section 12 and being in communication with the inflation lumen 21 of the balloon catheter through small vents as shown in FIG. 10c. The tube 300 is made of a force sensitive material that changes color when a specified stress or pressure is achieved. When the internal pressure rises during inflation, the tube 300 may deform to create a bulge 305 (see FIG. 10b) that, if severe enough, will initiate a color change in the tube 300). Alternatively, the tube 300 may change color without a physical deformation.

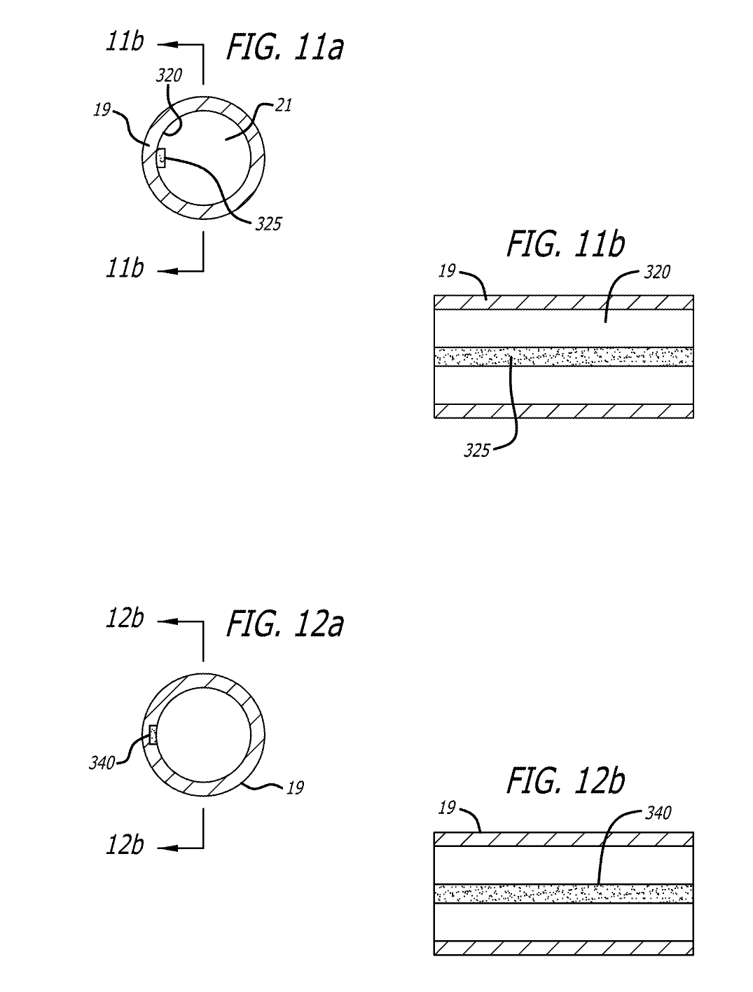

FIG. 11a illustrates yet another embodiment of the present invention wherein a portion of the inner surface 320 of the inflation lumen 21 contains a strip 325 of force sensitive material such as a mechanophore that extends along the longitudinal length of the inflation lumen, as shown in the cut-away view of FIG. 11b (Note the other components internal to the inflation lumen have been omitted for clarity). The strip 325 of force sensitive material will change colors if adhered to the inner surface 320 so as to stretch when the inner surface stretches, providing an indication of the pressure (and stress) experienced by the inflation lumen 21.

Alternatively, as shown in FIG. 12, the strip of material 340 can be embedded into the lumen tubing 19 rather than attached to the surface, reducing the possibility that it could be dislodged or loosened from the inner surface 320. In this case, the tubing 19 is formed with a groove sized to fit the strip of force sensitive material 340 such that it forms a flush surface on the tubing's interior. When the inflation lumen experiences pressurization during the inflation process, the strip of mechanophores will be stressed with the lumen material, and change color if the stress reaches a predetermined level. The catheter can be cut open in the event of a failure post-surgery to evaluate what the stress levels were in the inflation lumen during the procedure.

In each of the embodiments using a force sensitive material such as one including mechanophores, the various indicators can be used with multiple pressure sensing devices that measure different levels, so that a more accurate assessment can be made of the pressure experienced by the lumen. For example, by using one mechanophore that changes color at one stress level and a second mechanophore that and others are not the level of pressurization can be more accurately determined.

The structure includes material that reacts to a specific stress by changing color. Descriptions of such materials can be found in articles such as “Mechanical Stress Leads To Self-Sensing In Solid Polymers,” Biochemistry, May 6, 2009, found at http://www.phvsorg.com/news160834918.html, and “Development Of Polymer Films That Change Color In Response To Tension,” AIST press release of Oct. 7, 2008, found at http://www.aist.go.ip/aist_e/lastest research/2008/20081117/20081117.html. From the latter, such material can be formed by polymerization of acetylene substituted with a substituted phenyl group using [Rh(norbornadiene)Cl]2 as a catalyst to produce a polymer in which the main chain is in the cis conformation and has a helical structure. A film of this polymer may be prepared by spin-coating from a chloroform solution of the polymer on a colorless elastic sheet. The color of the obtained film of the substituted polyacetylene is, for example, yellow at the time of formation. By stretching this film together with the sheet by using a stretching machine, the substituted polyacetylene molecules were oriented along the direction of stretching. Further stretching led to a color change in the film from yellow to red. Measurement of the ultraviolet-visible absorption spectrum indicated an increase in absorption from approximately 500 to 600 nm. Removal of the tension and contracting the film led to the return of the color of the film from red to yellow, and the absorption spectrum agreed with the spectrum before stretching. Thus, the change in color due to stretching and contracting was reversible.

The color of the film changes instantaneously in response to quick manual stretching and contraction. The change in color is repeatable, that is, the change in color between yellow and red can be repeated by stretching and contraction repeatedly. In addition, the change in color is not dependent on the stretching ratio but on the tension applied. This color change is believed to be the result of a change in the conjugated system of the main chain due to the change in length of the polymer molecules associated with the stretching and contraction of the film.

Films prepared from other substituted polyacetylenes also showed instantaneous reversible changes in color between colorless and yellow or between purple and blue. These changes in color were also repeatable. However, when polymer was stressed to failure, the color change remained in the specimen, indicated that the designated pressure threshold had been reached. Thus, it can be used as a forensic tool to evaluate the pressure or stress of the material at failure and provide feedback on the conditions of the balloon catheter at the time of failure.

While the present invention is described herein in terms of certain preferred embodiments, those skilled in the art will recognize that various modifications and improvements may be made to the invention without departing from the scope thereof. Moreover, although individual features of one embodiment of the invention may be discussed herein or shown in the drawings of the one embodiment and not in other embodiments, it should be apparent that individual features of one embodiment may be combined with one or more features of another embodiment or features from a plurality of embodiments.

What is claimed:

1. A balloon catheter, comprising:
a) an elongated catheter shaft having a proximal end, a distal end, and an inflation lumen;
b) an inflatable body in fluid communication with the inflation lumen; and
c) a visual indicator for evaluating whether a predetermined pressure in the inflatable body has been exceeded.

2. The balloon catheter of claim 1, wherein the visual indicator is located in a portion of the catheter visible to a practitioner while the distal end of the catheter is in a patient.

3. The balloon catheter of claim 2, wherein the visual indicator is located in a luer at the proximal end of the catheter.

4. The balloon catheter of claim 1, wherein the visual indicator comprises a force sensitive polymer that changes color when experiencing a specified stress.

5. The balloon catheter of claim 4, wherein the force sensitive polymer is a mechanophore.

6. The balloon catheter of claim 5, wherein the mechanophore forms a part of the catheter.

7. The balloon catheter of claim 6, wherein the mechanophore is located within a luer on the catheter.

8. The balloon catheter of claim 6, wherein the mechanophore is part of a chassis of the catheter.

9. The balloon catheter of claim 1, wherein the visual indicator is a portion of the catheter designed to permanently deform upon a pressure exceeding a maximum prescribed inflation pressure.

10. The balloon catheter of claim 9, wherein the catheter is designed to inflate and deflate upon the portion's permanent deformation without any loss of function.

11. The balloon catheter of claim 1, wherein the visual indicator includes a chamber wall exposed to an internal pressure of the catheter, wherein the chamber wall flexes to a position upon experiencing a predetermined pressure, said position contacting an indicator for providing indication of the exceeding of the predetermined pressure.
